(19) United States
(12) Patent Application Publication
(10) Pub. No.: US 2006/0127430 A1
(43) Pub. Date: Jun. 15, 2006

(54) Controlled-Release of Cosmetic and Pharmaceutical Agents via Osmotic Nano-Diffusion from Zeolite Cage Complexes

(75) Inventor: Shyam K. Gupta, Scottsdale, AZ (US)

Correspondence Address: SHYAM K. GUPTA;BIODERM RESEARCH, 5221 E. WINDROSE DRIVE, SCOTTSDALE, AZ, 85254, US

(73) Assignee: BIODERM RESEARCH, Scottsdale, AZ (US)

(21) Appl. No.: 11/307,824

(22) Filed: Feb. 24, 2006

Related U.S. Application Data
(63) Continuation-in-part of application No. 10/418,495, filed on Apr. 18, 2003.

(51) Int. Cl. A61K 8/26

(52) U.S. Cl. 424/401; 424/62

(57) ABSTRACT

The present invention discloses the application of cage complexes of zeolites for topical controlled delivery of organic cosmetic and pharmaceutical active agents. The treatment and protection of skin surface requires that certain organic cosmetic and pharmaceutical active agents be delivered slowly and allowed to remain on skin surface for extended periods. Zeolite cage complexes of the present invention do not absorb into skin and affect a controlled release of cosmetic and pharmaceutical agents by an osmotic nano-diffusion method upon contact with water. Topical treatments that includes skin aging, anti-wrinkle, antioxidants, skin whitening, acne, rosacea, sun screens, UV blocks, anesthetics, skin soothers, anti-irritants, anti-inflammatory agents, vitamins, hormones, and such that require a controlled release of active agents are now practical by the present invention.

This is a continuation-in-part of patent application Ser. No. 10/418,495 filed Apr. 18, 2003.

The present invention discloses the application of cage complexes of zeolites for topical controlled delivery of organic cosmetic and pharmaceutical active agents. The treatment and protection of skin surface requires that certain organic cosmetic and pharmaceutical active agents be delivered slowly and allowed to remain on skin surface for extended periods. Zeolite cage complexes of the present invention do not absorb into skin and affect a controlled release of cosmetic and pharmaceutical agents by an osmotic nano-diffusion method upon contact with water. Topical treatments that includes skin aging, anti-wrinkle, antioxidants, skin whitening, acne, rosacea, sun screens, UV blocks, anesthetics, skin soothers, anti-irritants, anti-inflammatory agents, vitamins, hormones, and such that require a controlled release of active agents are now practical by the present invention.

A delivery system is a stratagem to optimize the efficacy or aesthetics of a cosmetic or pharmaceutical product. For example, the medicine part of a cough syrup is usually bitter in taste. The addition of sweeteners and flavor enhancers to such compositions makes them palatable. Sweetened and flavored cough syrups are thus delivery systems for the bitter medicine that is also included in such compositions.

For active agents desirable for topical delivery, there is usually a belief that a delivery system for faster absorption of such agents into the skin is better. Although that may be applicable to certain active agents it is not universally desirable. For example, the protection of skin surface from harmful effects of UV and free-radicals requires that such protective active agents, such as sun screens and antioxidants, be delivered to the skin surface and allowed to remain on the skin surface for as long as possible before those agents are absorbed deeper into the skin and carried away into the bloodstream. A number of other active agents, such as topical anesthetics, skin whitening agents, topical antibacterials, anti-wrinkle agents, skin smoothing agents, and hydroxy acids can also benefit from their controlled topical delivery.

The prior art is abundant in disclosures that improve, enhance, or accelerate the absorption of skin and body beneficial active agents into skin. There is a general lack of suitable methods that can provide controlled topical delivery of skin, body, and hair beneficial active agents that can remain on the skin surface for extended periods of time.

It is the purpose of the present invention to disclose a new method for topical controlled-release of cosmetic and pharmaceutical active agents by osmotic-controlled nano-diffusion based on anionic zeolite cage complexes.

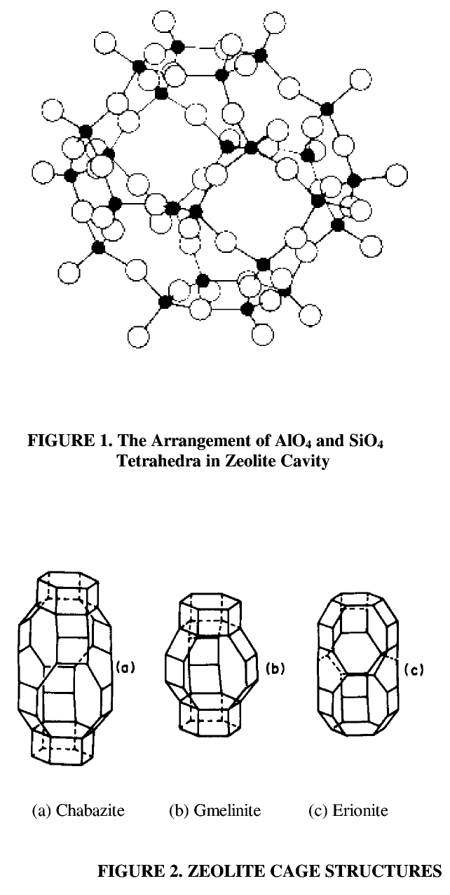

Zeolites are a group of crystalline aluminosilicates that have a porous, cage-like structure with a cavity. A zeolite may be defined as an aluminosilicate with a framework structure enclosing cavities occupied by large ions and water molecules, both of which have considerable freedom of movement, permitting ion-exchange and reversible dehydration. The framework consists of an open arrangement of corner-sharing tetrahedral where SiO4 are partially replaced by AlO4 tetrahedra, which requires sufficient cations to achieve electro neutrality [FIG. 1].

[FIG. 1].

There are some 37 natural and over 100 synthetic zeolites, the latter all made by hydrothermal synthesis. The main uses are as molecular sieves, catalysts, and catalyst support for platinum group metals. Zeolite cavities are usually occupied by water. Some typical cavities occurring in Zeolite cages are shown in FIG. 2.

[FIG. 2].

Dehydration of synthetic zeolites leaves cubic micro crystals in which A104 and SiO4 tetrahedra are linked together to form a ring of eight O atoms on each face of the unit cube and an irregular ring of six O atoms across each corner. In the center of the unit cell is a large cavity about 11.4 Angstroms in diameter, which is connected to six identical cavities in adjacent unit cells by the eight-membered rings, which have inner diameter of about 4.2 Angstroms. In addition, the large cavity is connected to eight smaller cavities, about 6.6 Angstroms in diameter, by the six-membered rings, which provide openings of about 2.0 Angstrom in diameter. In the hydrated form all the cavities contain water molecules. In the anhydrous state the same cavities may be occupied by other molecules brought into contact with the zeolite, provided such molecules are able to pass through the apertures connecting cavities. Molecules within the cavities then tend to be held there by attractive forces of electrostatic and van der Waals types. Thus the zeolites will be able to absorb and strongly retain molecules just small enough to enter the cavities. It will not absorb at all those too big to enter. It will absorb weakly very small molecules than can enter or leave easily, except water molecules, which bind strongly.

It is another purpose of this invention to utilize such Zeolite cavities for the formation of Zeolite cage complexes with organic active agents that may even be larger than zeolite cavities and their application to skin controlled topical deposition of such active agents.

The preparation and properties of zeolites are described in detail in U.S. Pat. No. 2,882,243, among other sources. Generally, the preparation involves combining aqueous solutions that are sources of silica, alumina and sodium to produce a gel that crystallizes upon hydrothermal treatment. Conventional washing and drying steps provide hydrated Zeolite Na. The hydrated Zeolite Na must be modified with the substitution of potassium for part of the sodium to form Zeolite K prior to activation. The potassium modification is carried out by ion exchange in aqueous solution using nearly any appropriate potassium salt such as potassium chloride, potassium nitrate, potassium sulfate, and the like. The exchange can be carried out in any convenient manner that allows control of the amount of potassium exchanged for sodium, or for sodium with other metals. Heating of the hydrated Zeolite K to a temperature above about 300° C. provides a zeolite that has a strong heat of hydration. It is another object of the present invention to utilize commonly available anionic zeolite as a method for depositing active agents on skin at a controlled rate.

The present invention discloses a method for the formation of cage complexes of organic active agents with anionic zeolites, i.e. natural or synthetic zeolites that have not been modified by chemical bonding with other organic reagents to change either their electrical charge properties, i.e. cationic or zwitterionic, or the size of their cavity or outer pores.

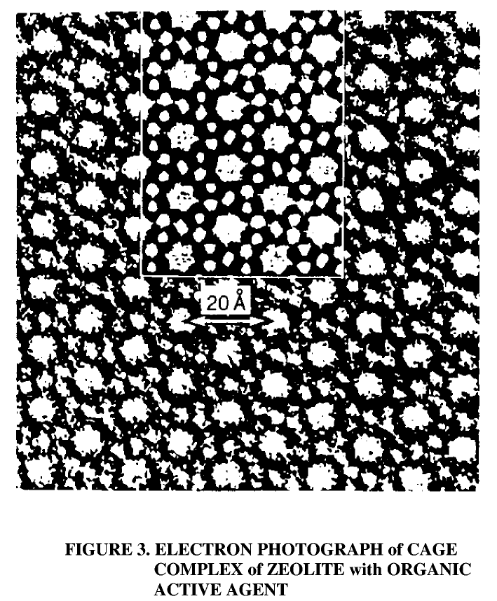

The cage complexes of organic active agents with anionic zeolites of the present invention provide an unexpected and surprising topical controlled release of said organic active agents upon the contact of said cage complexes with water molecules. The water molecules may be applied externally, or provided by the natural perspiration of skin. The cage complexes are prepared by contact of anionic zeolite with organic active agent. It is preferred to utilize an anhydrous form of the anionic zeolite. The organic active agent enters the zeolite cavity and forms a cage complex, the electron microscope photograph of one of such anionic Zeolite is shown in FIG. 3.

[FIG. 3].

Zeolites can be made with both specific pore structures and bound cations that have found applications in various self-warming cosmetic compositions in the prior art. U.S. Pat. No. 3,250,680 (Menkart et al.) discloses applications of Zeolites for the preparation of self-heating toothpaste and other such compositions. Menkart utilizes only the heat releasing property of zeolites.

U.S. Pat. No. 5,476,660 (Somasundaran et al.) discloses certain compositions of chemically modified zeolites in which zeolite surface has been modified to a positively charged state (cationic) or a zwitterionic state. These chemically modified zeolites have a filamentous structure with outwardly protruding positively charged organocarbonyl groups and also outwardly protruding negatively charged organocarbonyl groups. These chemically modified zeolites are useful for the deposition of active agents, more specifically, anionic active agents (column 3, line 21-23), thus limiting their utility. Somasundaran et al. do not disclose the deposition of active agents on skin using conventional anionic zeolites. Somasundaran et al. further disclose (column 5, line 39-52) the use of zeolite as carrier particles for depositing an active substance onto a target surface (claim 14). However, further examination of the teaching of Somasundaran et al. (column 5, line 39-52) reveals that zeolite is first chemically reacted or engrafted with one or more polymers that contain difunctional organocarbonyl groups, wherein one of the groups serves to attach the polymer to the zeolite surface and the second group serves as the functional group on thus attached organocarbonyl group, thus changing the electrical charge on zeolite from an anionic to a cationic or zwitterionic state. Unlike the anionic zeolite particles, the zwitterionic or cationic particles remain on the target surface even after the target surface has been washed off, which is a disadvantage for many applications in cosmetics—albeit such a property of zwitterionic or cationic particles being of possible advantage in laundry applications. Although the zwitterionic zeolite may have a negative charge under neutral pH (i.e., “a net zeta potential”), they still deposit substantially better than anionic particles with the same net negative charge, according to Somasundaran. Quite contrary to Somasundaran, the anionic zeolites do provide an excellent, highly desirable topical controlled-release of organic active agents, according to the present invention. The present invention requires only two agents; an anionic zeolite as the cage complex forming agent and an organic active agent that needs to be deposited on skin by a controlled-release method. Somasundaran et al. teachings require a combination of at least three agents [col. 18, line 46-50; claim 14, claim 15; which refer to col. 17, line 26-46; claim 1, sub-parts (i), (ii), and (iii)] to deposit an active substance onto skin: a carrier particle such as a zeolite, a coating or covalent bond forming agent such as a polymeric polyampholyte that converts zeolite into a cationic or zwitterionic state, and an active agent. An optional but highly preferred ingredient to be included in Somasundaran compositions (column 7, line 52-61) is a cationic or amphoteric polymer deposition aid. This requirement for an additional ingredient further limits the utility of Somasundaran teachings. Somasundaran et al. further disclose (column 7, line 35-42) that the active ingredient is deposited on the target surface along with the cationic carrier particles and the active is subsequently gradually released. The release of the active is accomplished by sheer (e.g., rubbing of the particles onto the skin), temperature (e.g., melting of the particles at body temperature), diffusion and combination thereof. The release of active agents in thus not controlled. In Somasundaran invention the cationic particles or filaments that are attached to carrier zeolite are also released at the same time, which may be a very serious problem as it may affect the efficacy of certain active agents being deposited topically concurrently. The present invention requires water molecules for the release of organic active agents from the cage complex of anionic zeolite and the organic active agent. The rate of controlled release is thus regulated by osmotic entry of water. The compositions of Somasundaran et al. are especially useful for depositing anionic actives. The present invention does not have any such limitations. Also, Somasundaran et al. compositions [claim 1, subpart (iii); claim 10, subpart (iv)] must also contain from 1% to 99% by weight of the composition of an anionic surfactant. As is well known in the prior art that the presence of such surfactants can actually impact the deposition of many organic active agents on skin. Moreover, such a large amount of surfactant can cause serious skin irritation. Somasundaran et al. further teach (claim 10, subpart (iii) the need for a cationic or amphoteric deposition aid, in combination with a modified zeolite carrier particles and the organic active agent, all of which make Somasundaran teachings even more complex and impractical, compared to the simple anionic zeolite—organic active agent cage complex controlled-release osmotic topical delivery method of the present invention. This detailed discussion of Somasundaran et al. teachings is considered pertinent due this reference kindly brought to the attention of the present inventor by the examiner of an earlier disclosure (U.S. patent application Ser. No. 10/418,495, filed Apr. 18, 2003; now abandoned).

U.S. Pat. No. 6,752,998 (Verdrel-Lahaxe et al.) discloses an exothermic composition, which includes at least one zeolite; at least one surfactant; at least one magnesium or calcium halide; and a physiologically acceptable anhydrous medium. Verdrel-Lahaxe et al. utilize only the heat-releasing or rubefacient properties of zeolites and do not disclose any controlled topical release of active agents.

U.S. Pat. No. 4,626,550 (Hertzenberg) discloses certain personal care products such as lotions and creams that are prepared using potassium exchanged Zeolite A that is much less anionic in nature. These compositions are useful only for the release of heat, and the inclusion of active agents such as bodying agents, topical pain relievers, antiperspirants and others must be largely anhydrous and should not enter the structures of the zeolite to release heat (col. 3, line 50-57). Hertzenberg does not disclose any controlled topical release of such active agents.

U.S. Pat. No. 4,379,143 (Sherry et al.) discloses activated or partially activated zeolites that can be included in analgesic balms or ointments as improved replacements for rubefacients. Upon hydration, the zeolite becomes warm, thereby helping to relieve pains associated with various musculoskeletal problems. Varying the character of the liquid vehicle can control generation and maintenance of the heat of hydration of anhydrous zeolite. If a very quick release of heat is desired, a hydrophilic vehicle is used; if a slow, sustained heat release is desired, a hydrophobic vehicle is required. Intermediate and controlled performance can be introduced by altering the hydrophobic vehicle to provide some hydrophilic characteristics. Sherry et al. utilize only the heat-releasing or rubefacient properties of zeolites and do not disclose any controlled topical release of active agents.

U.S. Pat. No. 6,274,128 (Bergman et al.) discloses an essentially anhydrous hair conditioning composition that comprises zeolites of specific pore size larger than the critical diameter of a water molecule and both the carrier molecules and the hair conditioner molecules that have molecular diameters larger than the largest average pore size of the micro porous materials. Bergman et al. utilize only the heat-releasing or rubefacient properties of zeolites and do not disclose any controlled topical release of active agents.

U.S. Pat. No. 6,309,655 (Minnix) discloses a cosmetic composition comprising a self-heating component, self-indicating disintegrating granules comprised of water-insoluble polymer and a colorant, which gives users indications of the length of time the composition has been applied and the degree of mixing when in use. This application is thus aimed at self-heating properties of zeolites, and their length of heating effect. Minnix utilizes only the heat-releasing or rubefacient properties of zeolites and do not disclose any topical release of active agents.

U.S. Application 2001 001 6201 (Janchitraponvej) discloses a yet another self-heating application of an anhydrous rinse-out hair care composition utilizing zeolites.

Self-warming compositions have also been made with various anhydrous alkali metal salts (Giani et al., U.S. Pat. No. 5,747,004). In self-warming formulations based on Zeolites, the pore size specification is typically very small, from 3 to 10 angstroms in diameter, as is the ratio between sodium and potassium cations bound to silicate anions of such zeolites. These formulations release heat upon contact with water. Water penetrates the pores of such Zeolites and hydrates the interior silicate atoms of Zeolite agglomerates. Such interaction of zeolite with water releases the heat of hydration. Most cosmetic lotion, cream, shampoo, and conditioner products also contain hydrophilic and lipophilic ingredients for skin and hair care benefits. Some of such ingredients tend to clog the pores of Zeolites, causing a reduction in the heat-release properties of such formulations. The examples of such fatty materials that can inhibit the heat release properties of zeolites include most surfactants used in shampoo and body wash applications; quaternary ammonium compounds used for hair conditioning applications; fatty esters used as emollients in skin lotion and cream applications, and other similar examples. While such clogging of zeolite pores by above mentioned ingredients was considered a problem, some of which are highly desirable active agents, and those problems were solved in the prior art by the use of small pore size zeolites that permit the entrance of water molecules inside their cavity but not other larger size molecules, for example U.S. Pat. No. 6,274,128, the prior art did not recognize that the interaction of such ingredients with zeolite was actually resulting in the formation of zeolite—active agent cage complexes and not clogging of zeolite pores. The present invention is first to recognize said cage complex formation and its utility in controlled topical delivery of active agents that are bound in such cage complexes upon the contact of cage complex with water.

U.S. patent application Ser. No. 20050133049 (Fournier et al.) discloses filters, smoking articles, and methods for selectively removing one or more selected constituents from mainstream smoke. The filters comprise zeolite BETA. Fournier et al. did discover that certain organic agents can bind with zeolite, but they failed to utilize this knowledge in the development of methods for topical delivery of such zeolite bound agents.

U.S. patent application Ser. No. 20050058597 (Corbin et al.) discloses a process to synthesize nano-size Zeolite-A from an amorphous gel precursor with particle sizes of about 150 nm. The nano-sized Zeolite-A has been used for detergents. Corbin et al. did not disclose the utility of such nano-sized zeolites in controlled topical delivery of organic active agents.

It is worthy of note that although zeolites with many different cations, such as titanium, zinc, manganese, iron, and copper have been disclosed, any applications of such metal zeolites in topical delivery of active agents have not been disclosed. This lack of knowledge is of special importance, since zeolites with enhanced ion-exchange capacity are well known (U.S. Patent Application 20010053741, Mikko et al.; U.S. Pat. No. 5,935,891; Prior). U.S. Pat. No. 6,503,740 (Alther et al.) discloses zeolites treated with an organic modification compound such as quaternary amines, pyridinium compounds, and phosphonium amines that are useful for water treatment applications. U.S. Pat. No. 6,365,130 (Barry et al.) discloses zeolites exchanged with antimicrobial metals for a chewing gum application, or a laundry application (U.S. Pat. No. 6,454,813; Chan). Modified zeolites have been used for topical cancer therapy (U.S. Pat. No. 6,288,045; Kaufman).

Zeolites have a very large surface area that is ionic in its nature. This surface area covers both the outside of zeolite and the inside of zeolite's porous cavity. The size of the pores of this cavity determines the size of any molecules that can enter zeolite's internal cavity. Almost all prior art disclosures have focussed on the cavity of zeolite. Since molecules larger in size than zeolite's cavity cannot enter zeolite's internal surface area, the delivery of such molecules from zeolite has not been disclosed in the prior art. The present invention circumvents this difficulty, and it is now possible to provide a controlled topical release of organic active agents that may even be larger in size than the cavity of zeolite. This is because of the new complex formation method disclosed in the present invention that also allows the formation of a cage complex of an anionic zeolite with an organic active agent that is larger in size than zeolite pore; said cage complex is now being formed with the outer anionic surface of said zeolite when said organic active agent is in contact with said surface of said zeolite. As electron photographs have shown, some parts of said larger size organic active agent do enter the cavity of zeolite to form a cage complex, while other parts of said organic active agent remain attached to the outer surface of zeolite. It is like an octopus, which can enter its longer arms into the cavity of a submerged rock to extract a prey, while the main part of octopus remains on the outside of that rock. This property of zeolite-organic active agent cage complex was not known to the prior art, as it became known to the present inventor mostly due to the availability of the electron photograph shown in FIG. 3.

I have now found that zeolite's surface area can be used for the controlled delivery of organic active agents. Moreover, the delivery of such molecules can be controlled by a osmotic-controlled delivery to provide a slow or sustained release of such agents for longer-term benefits. It is theorized at this point that the ionic nature of zeolite's surface can bind with organic molecules of certain molecular dimension in various modes such as ionic bond, ion-pair bonding, electrostatic attraction, Van der Waal's attraction forces, or Hydrogen bonding to form a zeolite cage complex with such agents. Upon contact with the outer layers of skin such agents remain bound as the zeolite cage complex until the moisture from skin perspiration or topically applied water enters zeolite cage complex. At this point organic active agents bound with zeolite as the cage complex diffuse from zeolite cage to skin surface. This delivery or release of organic active agents is osmotic controlled, as it depends on the rate of perspiration on skin or the rate of topical application of water and the rate of the entry of such perspiration into zeolite cage complex. Zeolite surface has a greater propensity to bind with water than the organic active agent that is bound as a cage complex with it.

The present invention also provides the first application of anionic zeolites as nano-delivery method in which the delivery of cosmetic and pharmaceutical agents is performed by osmotic-controlled nano-diffusion: the ingredients are delivered to skin surface by diffusion through the nano-pores of zeolite as single molecules from the large surface area of nano-sized zeolites to which such ingredients are bound as cage complexes; the diffusion itself being controlled by the rate of contact of water that is either applied topically or from skin perspiration.

The method of cage complex deposition on skin is preferably performed in the presence of a solubilizing agent, if the active agent for cage complex is a solid at ambient temperature. However, it is not always necessary to use a solubilizing agent if the active agent for the cage complex is a liquid at ambient temperature. Solubilizing agent is a water-miscible organic liquid selected from alcohol, ethylene glycol, diethylene glycol, triethylene glycol, polyethylene glycol; propylene glycol, dipropylene glycol, polypropylene glycol; butylene glycol, glycerol, ethylhexylglycerin, ethoxydiglycol, methylpropanediol, diglycerol, higher polyglycerols; sugar alcohols, adducts of glycerol with ethylene oxide or propylene oxide; adducts of sugar alcohols with ethylene oxide or propylene oxide; galactose, fructose, ethylene oxide adducts of galactose, propylene oxide adducts of galactose, ethylene oxide adducts of fructose, propylene oxide adducts of fructose, maltose, lactose, N-methylpyrrolidone, polyoxyethylene methylglucosides and a mixture thereof.

It is worthy of note that when the solubilizing agent is an ester of a hydroxy acid, then the method of the application of zeolite—active agent cage complex results in enhanced penetration of said active agent, as disclosed by Gupta (U.S. patent application Ser. No. 10/904,665; filed Nov. 22, 2004; claim 37). The examples of ester of a hydroxy acid include alkyl and aryl esters of Glycolic Acid, alkyl and aryl esters of Malic Acid, alkyl and aryl esters of Lactic Acid, alkyl and aryl esters of Mandelic Acid, alkyl and aryl esters of Ascorbic Acid, alkyl and aryl esters of Phytic Acid, alkyl and aryl esters of Salicylic Acid, alkyl and aryl esters of Aleuritic Acid, alkyl and aryl esters of Tartaric Acid, alkyl and aryl esters of Citric Acid, etc. As can be noted, the solubilizing agents of Gupta 10/904,665 are quite different in their properties than the solubilizing agents of the present invention. This also serves to illustrate that the properties of zeolite cage complex can be changed by the inclusion of other agents such as a different solvent or a surfactant.

A great variety of active agents of larger size can be deposited and bound to the outer surface of zeolites by the present invention. The examples include antiaging, anti-wrinkle, antioxidants, trace metal peptides, trace metal nucleotides, trace metal glycosides, body slimming, fat reduction agents, skin whitening, acne treatment, rosacea treatment, sun screens, UV blocks, anesthetics, skin soothers, anti-irritants, anti-inflammatory agents, vitamins, hormones, alkaloids, and such.

Examples of organic active agents to promote excess fat reduction, cellulite control, or toning benefits include Forskohlin extract (from Coleus forskohlii plant), Hydroxycitric acid, (from Garcinia cambogia, and plants of Garcinia family), L-Carnitine, Creatine, Human growth hormone (HGH), Chromium picolinate, Kola seed extract, Caffeine, Niacinamide, Psyllium husk, Chitosan, Lipoprotein complexes, Polyphenols, Gymnemic acid, Pyruvic acid and Pyruvate salts, salts of Hydroxycitric acid, Phaseolamin (from Phaseolus vulgaris extract), DHEA, Chitosan, Theophylline, Theobromine (or salts thereof such as Aminophylline), Roselle tea extract, Arabinose, Inosine, Adenosine, Fructose-1,6-diphosphate, Adenosine triphosphate (ATP), Adenosine diphosphate (ADP), Indomethacin, Baicalein, Extract of the plant of genus Tephrosia, Natriuretic peptide, Laminaria extract, Extract from berries of Panax genus plant, Gymnema sylvestre extract, 9-cis, 11-trans Conjugated linoleic acid and 10-trans, 12-cis conjugated linoleic acid isomers (conjugated linoleic acid, CLA), Synephrine, Hordenine, Octopamine, Tyramine, N-Methyltyramine, Azaftig, Extract of Climbing ivy (Hedera helix), Extract of Arnica (Arnica montana), Extract of Rosemary (Rosmarinus officinalis), Extract of Marigold (Calendula officinalis), Extract of Sage (Salvia officinalis), Extract of Ginseng (Panax ginseng), Extract of St. Johns-wart (Hypericum perforatum), Extract of Ruscus (Ruscus aculeatus), Extract of meadowsweet (Filipendula ulmaria), Extract of Orthosiphon (Ortosifon stamincus), and combinations thereof.

Examples of organic active agents to promote collagen and elastin include Ascorbic acid, Ascorbic acid derivatives, Glucosamine ascorbate, Arginine ascorbate, Lysine ascorbate, Glutathione ascorbate, Nicotinamide ascorbate, Niacin ascorbate, Allantoin ascorbate, Creatine ascorbate, Creatinine ascorbate, Chondroitin ascorbate, Chitosan ascorbate, DNA Ascorbate, Carnosine ascorbate, Vitamin E, various Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), α-Lipoic acid, Niacinamide lipoate, Glutathione, Andrographolide (Andrographis paniculata), Carnosine, Niacinamide, Potentilla erecta extract, Polyphenols, Grapeseed extract, Pycnogenol (Pine Bark extract), and combinations thereof. The quantities of such compositions can be safe and effective amounts as needed, and not limited to any specific limits.

Examples of skin beneficial hydroxy acid include salicylic acid, zinc salicylate, niacinamide salicylate, lactic acid, glycolic acid, malic acid, mandelic acid, ascorbic acid, ascorbyl phosphoric acid, hydroxycitric acid, hydroxytetronic acid, citric acid, niacinamide complexes of hydroxy acid, aleuritic acid, ellagic acid, rosmarinic acid, chlorogenic acid, polysulfonic acid, and hyaluronic acid (HYA). The quantities of such compositions can be safe and effective amounts as needed, and not limited to any specific limits.

Examples of organic active agents to whiten skin include hydroquinone, arbutin, hydroquinone derivatives, Paper Mulberry extract (Broussonetia kazinoke), Mitracarpe extract (Mitracarpus scaber), Bearberry extract (Arctostaphylos uva ursi), Yellow Dock extract (Rumex crispus and Rumex occidentalis), Glutathione, Leucocyte extract, Aspergillus orizae extract (Aspergillus orizae), Licorice Root extract (Glycyrrhiza glabra), Rosmarinic acid (Rosmarinus officinalis), Tetrahydrocurcumin, Green Tea extract (Camellia sinensis), Yohimbe extract (Pausinystalia yohimbe), Ecklonia cava extract, niacinamide, Hydroxytetronic acid, Spondias mombin extract, Maprounea guianensis extract, Walteria indica extract, Gouania blanchetiana extract, Cordia schomburgkii extract, Randia armata extract, Hibiscus furcellatus extract, and combinations thereof. The quantities of such compositions can be safe and effective amounts as needed, and not limited to any specific limits.

Examples of skin beneficial antioxidant organic active agents include Ascorbic acid, Ascorbic acid derivatives, Vitamin E, Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), alpha-Lipoic acid, Glutathione, Andrographolide, Grapeseed extract, Green Tea Extract, Polyphenols, Pycnogenol (Pine Bark extract), White Tea extract, Black Tea extract, (Andrographis paniculata), Carnosine, Niacinamide, Emblica extract, and combinations thereof. The quantities of such compositions can be safe and effective amounts as needed, and not limited to any specific limits.

Examples of UVA/UVB sunscreen agents include Galanga extract (Kaempferia galanga), Benzophenone-3, Benzophenone-4, Ethylhexyl Methoxycinnamate, Homosalate, Ethylhexyl salicylate, Octocrylene, Menthyl anthranilate, Avobenzone, Lawsone, Sulisobenzone, Trolamine salicylate, Lawsone, Glyceryl aminobenzoate, Cinoxate, PABA, Galanga, and combinations thereof. The quantities of such compositions can be safe and effective amounts as needed, and not limited to any specific limits.

Organic active agents to improve blood microcirculation are selected from Horse Chestnut Extract (Aesculus hippocastanum extract)), Esculin, Escin, Yohimbine, Capsicum Oleoresin, Capsaicin, Niacin, Niacin Esters, Methyl Nicotinate, Benzyl Nicotinate, Ruscogenins (Butchers Broom extract; Ruscus aculeatus extract), Diosgenin (Trigonella foenumgraecum, Fenugreek), Emblica extract (Phyllanthus emblica extract), Asiaticoside (Centella asiatica extract), Boswellia Extract (Boswellia serrata), Sericoside, Visnadine, Thiocolchicoside, Grapeseed Extract, Ginger Root Extract (Zingiber Officianalis), Piperine, Vitamin K, Melilot (Melilotus officinalis extract), Glycyrrhetinic acid, Ursolic acid, Sericoside (Terminalia sericea extract), Darutoside (Siegesbeckia orientalis extract), Amni visnaga extract, extract of Red Vine (Vitis-Vinifera) leaves, apigenin, phytosan, luteolin, and combinations thereof. The quantities of such compositions can be safe and effective amounts as needed, and not limited to any specific limits.

Examples of organic antimicrobial agents include Berberine, Triclosan, Triclocarban, various Tritons (quaternary ammonium compounds), Benzyl Alcohol, Dehydroacetic Acid, Phenoxyethanol, Ethylhexylglycerin, and combinations thereof. The quantities of such compositions can be safe and effective amounts as needed, and not limited to any specific limits.

Examples of vitamins include Vitamin A, Retinol, Retinoic acid, Tretinoin, members of Vitamins B group, Vitamin C, Vitamin D, Vitamin E, Vitamin K, Carotenes, Biotin, Folic Acid, and their derivatives, and combinations thereof. The quantities of such ingredients can be safe and effective amounts as needed, and not limited to any specific limits.

Examples of hormones include progesterone, androsterone, dehydroepiandrosterone (DHEA), Pregnenolone, androstenedione, melatonin, testosterone, and combinations thereof. The quantities of such compositions can be safe and effective amounts as needed, and not limited to any specific limits.

Examples of skin protectant drug agents include Allantoin, petrolatum, glycerin, dimethicone, urea, calamine, cocoa butter, kaolin, zinc acetate, zinc carbonate, and combinations thereof. The quantities of such compositions can be safe and effective amounts as needed, and not limited to any specific limits.

Examples of organic active agents include various trace metals delivery systems, which includes copper, zinc, and manganese in their complexed or chelated forms. The complexed forms include copper, zinc, or manganese derivatives of Adenosine Triphosphate, Benfotiamine, Pyridoxal-5-phosphate, and fructose-1,6-diphosphate.

Even the active agents that are difficult to penetrate topically, or are unstable, can be delivered in the controlled-release manner by the present invention. The examples include Retinol, Retinoic acid, Retene A, Phytantriol, Panthenol, Thiamine, Riboflavin, Niacin, Niacinamide, Vitamin C, Vitamin C derivatives, Pyridoxine, Biotin, Folic Acid, Coenzyme Q10, Lipoic Acid, and Hydroquinone.

### EXAMPLES

The following examples illustrate presently preferred practice thereof. As illustrations they are not intended to limit the scope of the invention, unless otherwise stated herein. All quantities are in weight %.

### Example 1

### A Method of Topical Treatment with an Organic Antiaging Active Agent at a Controlled Rate via Zeolite—Active Agent Cage Complex

The following steps are performed for this method of topical treatment. (1) The antiaging active agent 1.0% (the antiaging agent is an equal weight mixture of Tetrahydrocurcumin, Niacinamide Lactate, Copper ATP complex, Glutathione, and Carnosine) and Glycerin 49.0% are mixed together, then Sodium Potassium Aluminosilicate (anionic Zeolite, pore size 9 Angstroms) 20.0% is added to it and mixing continued. Some heat is given off at this stage and zeolite—antiaging active agent cage complex is formed. Magnesium Sulfate (Anhydrous) 30.0% is added to it as a skin feel mineral agent. (2) The cage complex is applied topically. (3) Upon entry of moisture from skin perspiration into said cage complex the cage complex dissociates and releases organic active agents into skin that provides desired topical treatment, and wherein the rate of such release is dependent on the rate of said entry of moisture from skin perspiration into said cage complex.

### Example 2

### A Method of Combining an Anionic Zeolite with Organic Active Agents to Form Cage Complex of Zeolite with Organic Active Agents with the Inclusion of a Solubilizing Agent

The following steps are performed. A combination of (1) PEG-6 50.0% (2) Vitamin A Palmitate 0.1% (3) Vitamin E Acetate 0.1% (4) Actiplex Botanicals 0.1% (5) Phenoxyethanol 0.5% (6) Parabens 0.2% (7) Niacinamide 0.5%, and (8) Hydroxypropyl cellulose 0.5%, is mixed at 40 to 50 C for 6 hours, then (9) anionic Zeolite (Atofina Nk30-pore size 13.0 Angstroms) 48.0% is added and mixing continued for an additional 2 hours. The zeolite has a pore opening at least 1.0 Angstrom unit larger than the three-dimensional molecular geometry of organic active agents, which allows the entry of organic active agents into zeolite cavity that form cage complex with zeolite.

### Example 3

### A Method to Control Topical Delivery of an Organic Active Agent

The following steps are performed. (A) A Cage Complex is formed by mixing (1) PEG-6 50.0% (2) Vitamin A Palmitate 0.1% (3) Vitamin E Acetate 0.1% (4) Arbutin 0.1% (5) Phenoxyethanol 0.5% (6) Chlorphenesin 0.2% (7) Resacetophenone 0.5%, and (8) Hydroxypropyl cellulose 0.5%, at 40 to 50 C for 6 hours, then (9) anionic Zeolite (Atofina Nk30-pore size 9.0 Angstroms) 48.0% is added and mixing continued for an additional 2 hours. The zeolite has a pore opening at least 1.0 Angstrom unit larger than the three-dimensional molecular geometry of organic active agents, which allows the entry of organic active agents into zeolite cavity that form cage complex with zeolite. (B) The cage complex is applied topically. (C) Water is applied at a controlled rate as desired. Upon entry of water into cage complex the cage complex dissociates and releases organic active agent onto skin, and wherein the rate of such release is dependent on the rate of entry of water into cage complex from such controlled application of water.

### Example 4

### A Method for Facial Treatment Via Controlled Release of Anti-wrinkle Active Agents from Zeolite—Active Agents Cage Complex

The following steps are performed. (A) A Cage Complex is formed by mixing (1) PEG-6 45.0% (2) Dimethicone 2.0% (3) Vitamin A Palmitate 0.001% (4) Vitamin E Acetate 0.001% (5) Resacetophenone 0.01% (6) Phenoxyethanol 0.5% (7) Parabens 0.2% (8) Huber 90 White Clay 14.0% (9) Magnesium Aluminum Silicate 2.0% (10) Copper ATP 0.1% (11) Glutathione 0.1% (12) Licorice Root Extract 0.5% at 40 to 50 C for 6 hours, then (13) anionic Zeolite (Atofina Nk30-pore size 9.0 Angstroms) 36.0% is added and mixing continued for an additional 2 hours. The zeolite has a pore opening at least 1.0 Angstrom unit larger than the three-dimensional molecular geometry of organic active agents, which allows the entry of organic active agents into zeolite cavity that form cage complex with zeolite. (B) The cage complex is applied topically. (C) Water is applied at a controlled rate as desired. Upon entry of water into cage complex the cage complex dissociates and releases anti-wrinkle active agents into skin, and wherein the rate of such release is dependent on the rate of entry of water into cage complex from said controlled application of water.

### Example 5

### A Method to Retard Topical Penetration of Organic Active Agents

The following steps are performed for this method of topical treatment. (1) A mixture of Hydroxypropyl cellulose 0.25%, Butylene glycol 25.838%, Methylpropanediol 10.0%, and PEG-69.9% is mixed at 80 to 90 C till a clear solution is obtained. (2) anionic Zeolite (pore size 4-5 Angstroms) 28.0%, Disodium Lauryl Sulfoacetate 5.0%, and Sodium Cocoyl Isethionate 111.5% are then added with mixing. The mixture is cooled to 40 to 45 C, and (3) a mixture of Superoxide dismutase activation agent 7.34% (Superoxide dismutase activation agent is a mixture of Phytoplenolin, Niacinamide Lactate, Niacinamide Glycolate, Niacinamide Malate, Copper ATP complex, Zinc ATP, Manganese ATP, Glutathione, and Carnosine) is added. (4) Vitamin A 0.001%, Vitamin E 0.001%, Fragrance 1.5%, Shea butter 1.0%, Apricot Kernel Oil 0.6%, Grapeseed oil 0.8%, Mango butter 0.6%, Phenoxyethanol 0.7%, Chlorphenesin 0.3% and Walnut shell powder 4.0% are then added and mixing continued. It is allowed to cool to ambient temperature. Some heat is given off at this stage and the cage complex of zeolite—Superoxide dismutase activation agent is formed in-situ. (5) The cage complex is applied topically. (6) Upon entry of moisture from skin perspiration into said cage complex the cage complex dissociates and releases Superoxide dismutase activation agent into skin at a retarded rate, the rate of such release is dependent on the rate of said entry of moisture from skin perspiration into said cage complex. (7) Water is applied and upon rubbing foam is generated, which is rinsed off. This treatment with water further releases active agents from said cage complex.

### BRIEF DESCRIPTION OF DRAWINGS

[FIG. 1]. The Arrangement of A104 and SiO4 Tetrahedra in Zeolite Cavity.

[FIG. 2]. Zeolite Cage Structures.

[FIG. 3]. Electron Photograph of Cage Complex of Zeolite with Organic Active Agent.

What is claimed is:

1. A method of topical treatment with an organic active agent, wherein said organic active agent is released into skin at a controlled rate, and comprising; (i) the contact of at least one anionic zeolite and at least one organic active agent to form a cage complex of said zeolite and said organic active agent, and (ii) said cage complex is applied topically, and (iii) upon entry of moisture from skin perspiration into said cage complex the cage complex dissociates and releases organic active agent into skin, and wherein the rate of such release is dependent on the rate of said entry of moisture from skin perspiration into said cage complex.

2. A method according to claim 1, wherein zeolite is selected from a group of aluminosilicates that can be either in hydrated or anhydrous forms.

3. A method according to claim 1, wherein said cage complex contains from 0.0001 to 50.0 percent by its weight of organic active agent(s).

4. A method according to claim 1, wherein organic active agent is selected from a vitamin, hormone, plant extract, skin whitening agent, anti-inflammatory agent, emollient, moisturizer, skin protectant, humectant, silicone, skin soothing agent, sun screen agent, analgesic, anesthetic, trace metal complex, and combinations thereof.

5. A method according to claim 1, wherein organic active agent is Vitamin E.

6. A method according to claim 1, wherein organic active agent is Resacetophenone.

7. A method according to claim 1, wherein organic active agent is selected from Copper Adenosine Triphosphate, or Zinc Adenosine Triphosphate, or Manganese Adenosine Triphosphate, or combinations thereof in various proportions.

8. A method according to claim 1, wherein a solubilizing agent is included, which is selected from alcohol, ethylene glycol, diethylene glycol, triethylene glycol, polyethylene glycol; propylene glycol, dipropylene glycol, polypropylene glycol; butylene glycol, glycerol, ethylhexylglycerin, ethoxydiglycol, methylpropanediol, diglycerol, higher polyglycerols; sugar alcohols, adducts of glycerol with ethylene oxide or propylene oxide; adducts of sugar alcohols with ethylene oxide or propylene oxide; galactose, fructose, ethylene oxide adducts of galactose, propylene oxide adducts of galactose, ethylene oxide adducts of fructose, propylene oxide adducts of fructose, maltose, lactose, N-methylpyrrolidone, polyoxyethylene methylglucosides and a mixture thereof.

9. A method according to claim 1, wherein a cosmetic base is included.

10. A method according to claim 4, wherein trace metal complex is selected from Copper Benfotiamine, or Zinc Benfotiamine, or Manganese Benfotiamine, or combinations thereof in various proportions.

11. A method of combining an anionic zeolite with an organic active agent to form a cage complex of zeolite with organic active agent, and comprising the mixing of an anionic zeolite with an organic active agent, and wherein said zeolite has a pore opening at least 1.0 Angstrom unit larger than the three-dimensional molecular geometry of said organic active agent to allow the entry of said organic active agent into zeolite cavity and form said cage complex with zeolite.

12. A method according to claim 11, wherein organic active agent is selected from a vitamin, hormone, plant extract, skin whitening agent, anti-inflammatory agent, emollient, moisturizer, skin protectant, humectant, silicone, skin soothing agent, sun screen agent, analgesic, anesthetic, trace metal complex, and combinations thereof.

13. A method according to claim 11, wherein a solubilizing agent is included, which is selected from alcohol, ethylene glycol, diethylene glycol, triethylene glycol, polyethylene glycol; propylene glycol, dipropylene glycol, polypropylene glycol; butylene glycol, glycerol, ethylhexylglycerin, ethoxydiglycol, methylpropanediol, diglycerol, higher polyglycerols; sugar alcohols, adducts of glycerol with ethylene oxide or propylene oxide; adducts of sugar alcohols with ethylene oxide or propylene oxide; galactose, fructose, ethylene oxide adducts of galactose, propylene oxide adducts of galactose, ethylene oxide adducts of fructose, propylene oxide adducts of fructose, maltose, lactose, N-methylpyrrolidone, polyoxyethylene methylglucosides and a mixture thereof.

14. A method according to claim 11, wherein a cosmetic base is included.

15. A method according to claim 11, wherein said cage complex is applied topically.

16. A method to retard topical penetration of an organic active agent, and comprising; (i) at least one anionic zeolite and at least one organic active agent are contacted together to form a cage complex of said zeolite and said organic active agent, and (ii) said cage complex is applied to skin, and (iii) water is applied to cage complex on the skin, whereupon water enters the cage complex and binds with zeolite moiety of said cage complex, and (iv) said binding of water with said zeolite releases said organic active agent, which exits from said zeolite pores and enters into skin, and (v) wherein the rate of retarded release of organic active agents is dependent on the rate of said application of water to said cage complex.

17. A method according to claim 16, wherein a solubilizing agent is included, which is selected from alcohol, ethylene glycol, diethylene glycol, triethylene glycol, polyethylene glycol; propylene glycol, dipropylene glycol, polypropylene glycol; butylene glycol, glycerol, ethylhexylglycerin, ethoxydiglycol, methylpropanediol, diglycerol, higher polyglycerols; sugar alcohols, adducts of glycerol with ethylene oxide or propylene oxide; adducts of sugar alcohols with ethylene oxide or propylene oxide; galactose, fructose, ethylene oxide adducts of galactose, propylene oxide adducts of galactose, ethylene oxide adducts of fructose, propylene oxide adducts of fructose, maltose, lactose, N-methylpyrrolidone, polyoxyethylene methylglucosides and a mixture thereof.

18. A method according to claim 16, wherein organic active agent is selected from a vitamin, hormone, plant extract, skin whitening agent, anti-inflammatory agent, emollient, moisturizer, skin protectant, humectant, silicone, skin soothing agent, sun screen agent, analgesic, anesthetic, trace metal complex, and combinations thereof.

19. A method according to claim 16, wherein said cage complex contains from 0.0001 to 50.0 percent by its weight of organic active agent(s).

20. A method according to claim 16, wherein a cosmetic base is included.
